(19) United States
(12) Patent Application Publication
(10) Pub. No.: US 2022/0248782 A1
(43) Pub. Date: Aug. 11, 2022

(54) A BIOSENSORY GARMENT

(71) Applicant: JJ1 Holdings Pty Ltd, Queensland (AU)

(72) Inventor: Jeanette Mary Jones, South Brisbane (AU)

(21) Appl. No.: 17/629,262

(22) PCT Filed: Oct. 12, 2020
(86) PCT No.: PCT/AU2020/000121

§ 371 Date: Jan. 21, 2022

(30) Foreign Application Priority Data
Oct. 25, 2019 (AU) 2019904017

(51) Int. Cl. A41D 13/12; A61B 5/00; A41D 13/00; A61B 17/135

(52) U.S. Cl. CPC A41D 13/1281; A61B 5/6804; A41D 13/0015; A61B 5/0022; A61B 5/6802; A61B 5/01; A41D 2400/12; A41D 2400/322; A41D 2600/10; A61B 2560/0462; A61B 5/0024; A61B 17/1355

(57) ABSTRACT

A biosensory garment comprising removal portions to permit access to parts of a wearer's body. The portions having physiological sensors in contact with the wearer's body. A central processor adapted to receive, process and transmit sensor information to a command computer Wherein the wearer's physical condition can be monitored or recorded over a time period to facilitate medical intervention, ongoing recovery or rehabilitation.

### FIELD OF THE INVENTION

This invention relates to physiological monitoring devices. In particular, it concerns a biosensory garment including a smart or intelligent functionality. More particularly, the garment typically worn as an undergarment assesses and communicates the physical condition of an athlete or soldier for the purposes of monitoring performance and health status in the interest of rapid and efficient delivery of medical attention and can facilitate the implementation of ongoing physical recovery and rehabilitation.

### BACKGROUND OF THE INVENTION

Studies have indicated that the majority of deaths on the battlefield are a result of wounded soldiers not receiving immediate medical attention within the “golden hour”. This is compounded by the fact that medics often have difficulty in actually locating injured personnel especially in dense vegetation or unfriendly or geographically challenging terrain. Immediate assessment of medical condition or the ability to treat an injured soldier via telemetric and remote means is therefore vital to the survival of the injured combatant.

In the case of athletic performance and recovery from injury, a device for ambulatory and real time monitoring of the athlete's physiology is an invaluable asset. Important vital signs to be monitored may include without limitation, electrocardiograms (ECG) and electroencephalograms (EEG), heart rate, blood pressure, respiration rate, oximetry or blood oxygen saturation, blood glucose level, skin perspiration, capnography, body temperature, proprioception, motion evaluation, and may include implanted cardiac devices such as pacemakers, as well as monitoring various ambient parameters such as air temperature and humidity.

In the case of traumatic injury for example where arterial bleeding is experienced, the ability to apply a tourniquet can be difficult if not improbable where the injured soldier is unconscious or does not have access to assistance or a medic in the vicinity.

Furthermore, delay in administering immediate medical assistance is particularly relevant in the successful treatment of brain injuries. The ability to monitor brain activity via an EEG system is invaluable in this context. This is well established in the case of strokes or transient ischemic episodes and more recently, in the ongoing treatment of sporting concussive injuries leading to chronic traumatic encephalitis. In a military context, it has been estimated that between a quarter and a third of Australian Defence Force personnel have experienced at least one Mild traumatic Brain Injury (MTBI) in their lifetime.

The ability to anticipate the type of medical treatment required prior to the actual rescue and evacuation of injured military personnel also has the immense benefit of simply saving time and ultimately, lives. This is naturally often dependent on the medical expertise and knowledge of the first responder.

It is thus of inestimable benefit to be able to accurately assess the immediate physical condition of the injured soldier in real time by telemetric or other means within what is commonly referred to as the “golden hour”. This is especially where the soldier may be in a state of unconsciousness, delirium or unable to verbally communicate with a central command post or fellow soldiers in the first hour of becoming injured.

In the sporting arena, the ability to monitor brain trauma is also becoming more important. In light of recent American football experience with increasing awareness of chronic traumatic encephalitis (CTE), other sporting bodies such as the Australian National Rugby League and Rugby Australia are starting to implement player safety strategies such as immediately removing from the field players suspected of concussive injury for a Head Injury Assessment (HIA) by an onsite team physician.

While this is an improvement in player safety and welfare, traumatic brain injury usually manifests itself at a later stage. It may be crucial in any ongoing concussion rehabilitation program to monitor brain function and activity by some form of ambulatory means. There is presently no convenient means to the knowledge of the applicant of ambulatory electro encephalogram (EEG) monitoring while a sportsman is away from a clinical environment and continues to train on the field.

It is therefore an object of the invention to seek to eliminate or ameliorate the problem(s) hereinbefore described by providing a biosensory garment wherein the physical condition of a user can be immediately assessed and monitored during ambulatory activity or incapacity.

It is a further object of the invention to provide a biosensory garment wherein the physical performance of a user such as an athlete can be monitored and which can be used to facilitate the implementation of any ongoing recovery or rehabilitation program.

### SUMMARY OF THE INVENTION

In a broad aspect, the invention resides in a biosensory garment comprising one or more wearable portions; the portions able to be attached together by releasable fasteners wherein the portions can be individually removed to permit access to various parts of a wearer's body; the one or more portions having at least one physiological sensor in contact with the wearer's body; a central processor adapted to receive signals from the at least one physiological sensor; the central processor to process and transmit sensor information to a command computer, wherein in operation, the physical condition of the wearer can be monitored in real time or recorded over a period of time by the command computer in facilitating any medical intervention or ongoing recovery or rehabilitation of the wearer.

Preferably, the biosensory garment is comprised of a light weight, breathable textile or fabric when used in warm and humid conditions.

In the alternative, the biosensory garment can be comprised of a thick or layered textile or fabric when used in cold and windy conditions.

Preferably, the biosensory garment comprises two wearable portions of a top or shirt portion and a bottom or pants portion.

In a more preferred example, there can be several wearable portions wherein the wearable portions are attached together with releasable fasteners including hook and eye fasteners, Velcro, press studs, magnetic studs or any other releasable fastening means. This arrangement enables one or more of the portions to be detached from adjacent portions to facilitate access to the user's body such as for the treatment of a wound or other injury.

Preferably, the one or more wearable portions comprises a close fitted hood or balaclava portion including EEG sensors adapted to be in contact with the head of the wearer.

In addition to EEG, the one or more physiological sensors can include ECG, body temperature, subcutaneous blood flow, blood pressure, oximetery, galvanic skin response or other non-invasive sensor.

Preferably, there is a transceiver receptive to signals from an implanted sensor such as a cardiac pacemaker and wherein the central processor transmits the signal information received by the transceiver to the command computer.

In a preferred embodiment, the one or more wearable portions include inflatable segments associated with an air supply and one or more microprocessor controlled air valves specially adapted to inflate and deflate and maintain inflation pressure of the inflatable segments to control blood flow or to operate as an occlusive tourniquet as required in the medical intervention or treatment of the wearer.

In a preferred example, the air supply comprises an air pump.

In the alternative, or in addition, the air supply can include a cylinder of compressed gas. Preferably, the central processor transmits information from the sensors to the command computer over a wireless personal network area protocol and wherein the command computer can comprise a remote server or smart device including a mobile phone, personal digital assistant, and laptop or tablet computer.

Preferably, there are one or more heating elements associated with the wearable portions to maintain or increase body temperature as required.

Suitably, there is a portable rechargeable battery power supply to operate the one or more physiological sensors, the central processor, any transceiver, any heating element, any air supply or microprocessor controlled air valve.

Suitably, the command computer coordinates and controls the operation of the one or more physiological sensors, any transceiver, any air supply or microprocessor controlled air valve or any heating element via the central processor.

In a preferred application, the biosensory garment is used as a military garment suitably worn under a uniform to facilitate the acquisition of vital signs and health status of combatants during normal operational duties and wherein the vital signs and health status information can also be used to anticipate any necessary medical intervention or treatment by first responders or medical prior to rescue and evacuation of an injured combatant.

In another application, the biosensory garment is used is used for the ambulatory acquisition of vital signs and health status monitoring external to a clinical environment over extended time periods in the recovery and treatment of an injured wearer.

The biosensory garment can also be an ambulatory sports activity and fitness monitoring device in the training and optimization of athletic performance and in the treatment and recovery of an athlete from a sports injury.

### BRIEF DESCRIPTION OF THE DRAWINGS

In order that the present invention is better understood reference is made to the accompanying drawings wherein:

FIG. 1 is a drawing of a preferred embodiment of the invention.

FIG. 2 is a drawing of a close fitted hood or balaclava portion with EEG sensors.

FIG. 3 is a drawing of the invention of FIG. 1 showing inflatable segments.

FIG. 4 is a drawing of the invention as used in a military application.

FIG. 5 is a drawing of the invention as used in an industrial application.

FIG. 6 is a drawing of the invention as used in a sporting application.

### DETAILED DESCRIPTION OF THE EMBODIMENTS

While the preferred embodiment herein described refers to a military application, the same considerations apply to an example used in the training and optimization of athletic performance and the treatment and recovery from sports injuries.

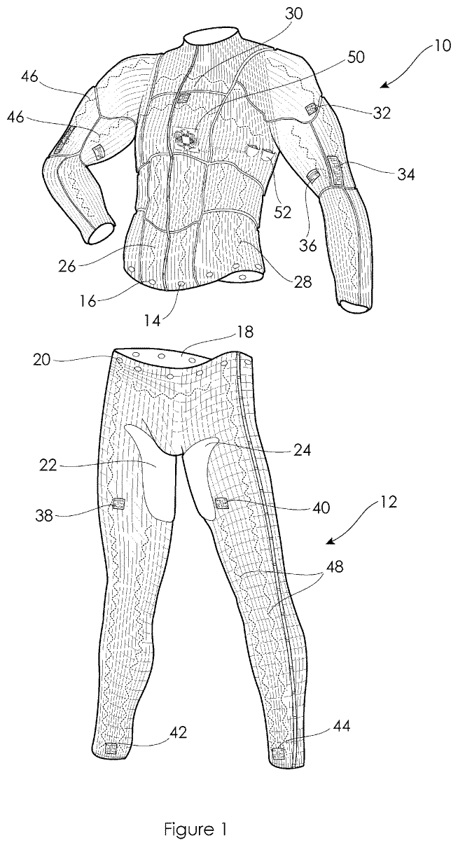

As shown in FIG. 1, the garment preferably comprises two wearable portions of a top or shirt portion 10 and a bottom or pants portion 12 attached together by releasable fasteners 14,16,18,20. In the alternative, it may comprise a one piece garment (not shown). There are detachable or removable sections 22,24 or panels 26,28 to allow access to the wearer's body.

As previously discussed, the garment is typically an undergarment and preferably comprises a light weight, breathable textile or fabric when used in warm and humid conditions or can be of a thick or layered configuration when used in cold and windy conditions. The releasable fasteners 14-20 can suitably include hook and eye fasteners, Velcro, press studs, magnetic studs or any other detachable fastening means. This arrangement enables one or more of the portions to be detached from adjacent portions to facilitate access to the user's body such as to treat wounds and injuries.

There are one or more physiological sensors which can include ECG 30, body temperature 32, subcutaneous blood flow 34, blood pressure 36, oximetery 38, galvanic skin response 40 or other skin contact non-invasive sensor 42, 44.

There is also one or more heating elements 46, 48 associated with the wearable portions to maintain or increase body temperature as may be required. The heating elements can be controlled by the central processor 50 which can itself be responsive to instructions from the command computer (not shown) to monitor and adjust the temperature of the one or more heating elements 46, 48.

Also shown is a transceiver 52 receptive to signals from an implanted sensor such as a cardiac pacemaker (not shown) and wherein the central processor 50 transmits the signal information received from the transceiver 52 to the command computer (not shown) over a wireless protocol. The command computer can comprise a remote server or smart device including a mobile phone, personal digital assistant, and laptop or tablet computer.

Medical information is then processed by the command computer and sent to a smart device carried by a first responder or medic treating the injured wearer. In the case of athletic training or rehabilitation, medical information can likewise be processed by the command computer and sent to smart device carried by a trainer or physiotherapist treating an injured athlete. It is anticipated that any ambulatory recovery, treatment or rehabilitation program can be implemented through the command computer and the central processor.

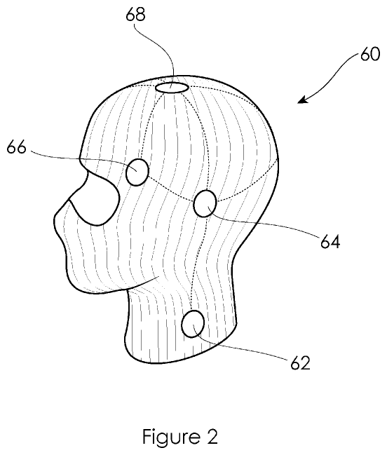

FIG. 2 shows a close fitted hood or balaclava portion 60 including strategically located EEG sensors 62-68 for contact with the head of a wearer.

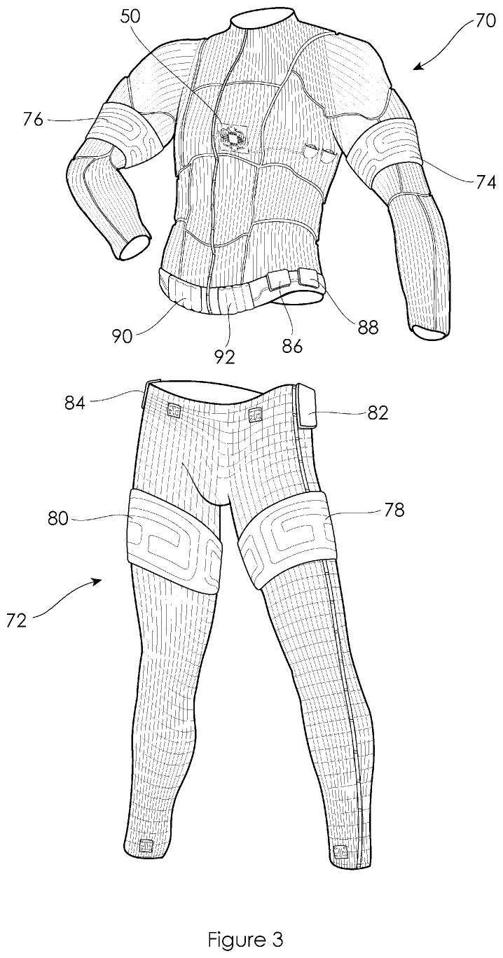

FIG. 3 is an example of the garment of FIG. 1 wherein the one or more wearable portions 70, 72 include inflatable segments74-80 associated with air supply pumps 82-88. There are one or more microprocessor controlled air valves (not shown) specially adapted to inflate and maintain air pressure of the inflated segments 74-80. In this embodiment, the invention can be used to control blood flow or to operate as an occlusive arterial tourniquet if required. Suitably, there is portable rechargeable battery power 90, 92 to operate the various physiological sensors, central processor, transceiver, heating element, air supply pump or microprocessor controlled air valves. Through the central processor 50, the command computer (not shown) is able to coordinate and control the operation of the one or more physiological sensors, the transceiver, air supply pump, air valve or heating elements. It is obvious that any recovery, treatment or rehabilitation program involving the application of heat and/or pressure over time will be able to be implemented through the central processor under control of the command computer.

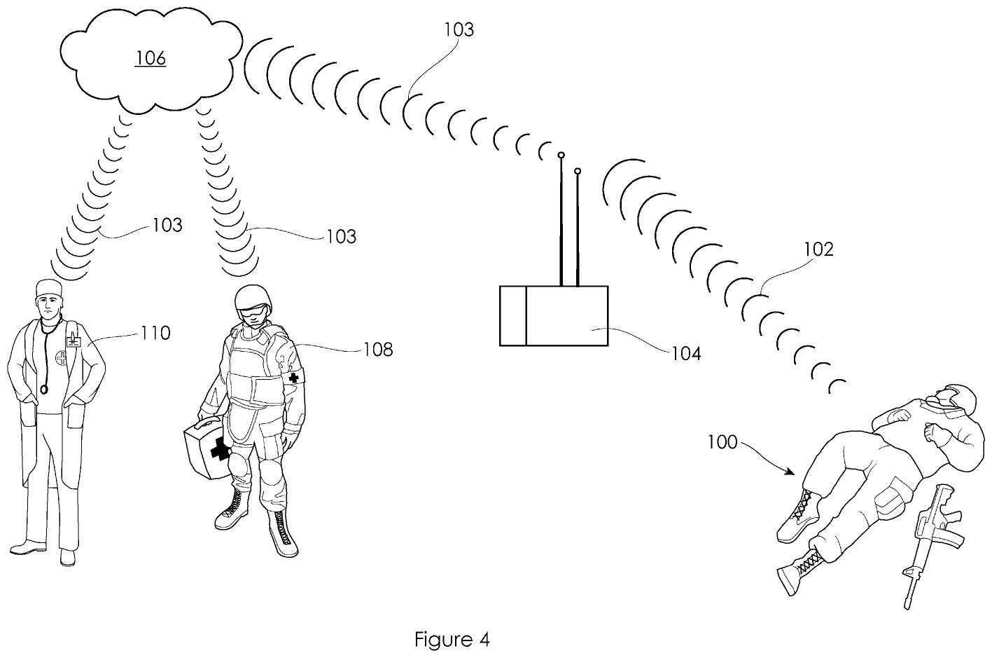

FIG. 4 shows an injured soldier 100 wearing the biosensory garment (not shown) as an undergarment under a battle uniform. In this application, the biosensory garment is used to facilitate the acquisition of vital signs and health status of combatants during normal operational duties and wherein the vital signs and health status information are sent 102 to a command computer 104 which in turn communicates 103 over a cloud network 106 to anticipate any necessary medical intervention or treatment by first responders or field medics 108 and military hospital staff 110 prior to rescue and evacuation of the injured combatant 100. As previously discussed, the biosensory garment can be used to facilitate the acquisition of vital signs and health status of combatants during normal operational duties.

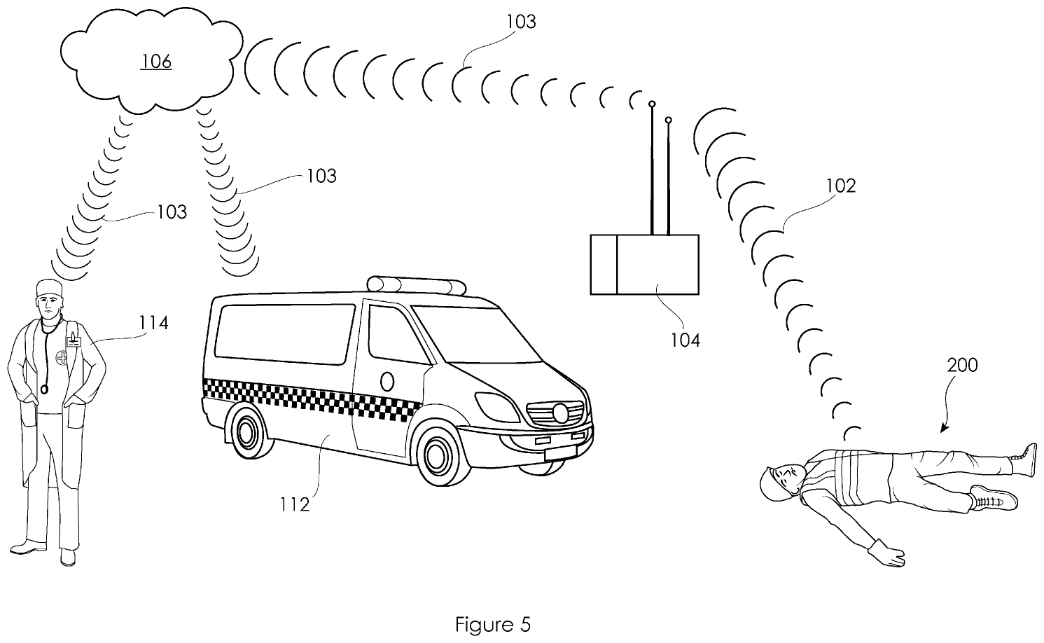

FIG. 5 shows another application, wherein the biosensory garment can be used for the acquisition of vital signs and health status monitoring in the recovery and treatment of an injured mine or construction worker 200. Also typically worn as an undergarment, the vital signs and health status information of the injured worker are sent 102 to a command computer 104 which communicates 103 over a cloud network 106 to smart devices carried in the ambulance 112 or by medical staff 114.

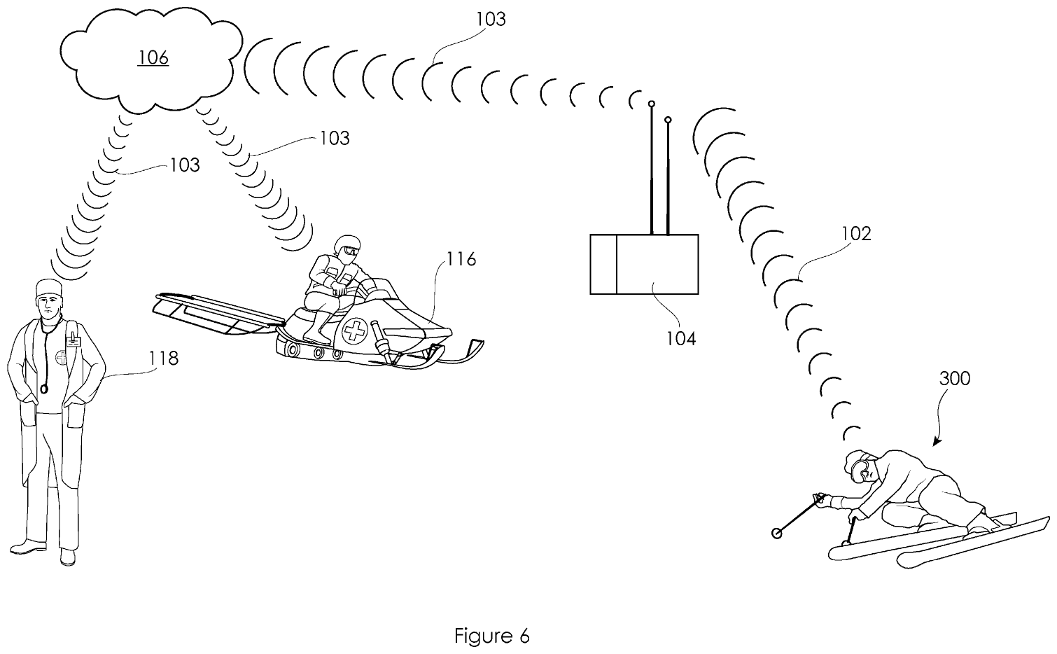

FIG. 6 shows a non-military context wherein the biosensory garment can be used as an ambulatory sports activity and fitness monitoring device in the training and optimization of athletic performance as well as in a treatment and recovery program in relation to a sports injury. In this example a skier 300 wearing the garment has his or her vital signs and health status information are sent 102 to a command computer 104 which communicates 103 over a cloud network 106 to anticipate any necessary medical intervention or treatment by first responders 116 and hospital staff 118 as part of a rescue and treatment protocol accordingly.

It will of course be realized that while the foregoing has been given by way of illustrative example of this invention and the best method of its utility known to the inventor, all such further and other modifications and variations thereto as would be apparent to persons skilled in the art are deemed to fall within the broad scope and ambit of this invention as herein set forth.

In the specification the terms “comprising” and “containing” shall be understood to have a broad meaning similar to the term “including” and will be understood to imply the inclusion of a stated integer or step or group of integers or steps but not the exclusion of any other integer or step or group of integers or steps. This definition also applies to variations on the terms “comprising” and “containing” such as “comprise”, “comprises”, “contain” and “contains”. It will also be understood that references to integers or elements defined in the singular likewise will also apply to their plural case and vice versa.

The invention as herein claimed is:

1. A biosensory garment comprising one or more wearable portions; portions able to be attached together by releasable fasteners wherein the portions can be individually removed to permit access to various parts of a wearer's body; the one or more portions having at least one physiological sensor in contact with the wearer's body; a central processor adapted to receive signals from the at least one physiological sensor; the central processor to process and transmit sensor information to a command computer, wherein in operation, the physical condition of the wearer can be monitored in real time or recorded over a period of time by the command computer in facilitating any medical intervention or ongoing recovery or rehabilitation of the wearer.

2. The biosensory garment of claim 1 comprising a single or one piece wearable portion.

3. The biosensory garment of claim 1 comprising two wearable portions of a top or shirt portion and a bottom or pants portion.

4. The biosensory garment of claim 1 comprising two or more wearable portions which are attachable together with releasable fasteners including hook and eye fasteners, Velcro, press studs, magnetic studs or any other releasable fastening means.

5. The biosensory garment of claim 1 wherein the one or more wearable portions comprises a close fitted hood or balaclava portion including EEG sensors adapted to be in contact with the head of the wearer.

6. The biosensory garment of claim 1 wherein the one or more physiological sensors include ECG, body temperature, subcutaneous blood flow, blood pressure, oximetery, galvanic skin response or other non-invasive sensor.

7. The biosensory garment of claim 1 including a transceiver receptive to signals from an implanted sensor such as a cardiac pacemaker and wherein the central processor transmits the signal information received by the transceiver to the command computer.

8. The biosensory garment of claim 1 wherein the one or more wearable portions include inflatable segments associated with an air supply and one or more microprocessor controlled air valves specially adapted to inflate and deflate and maintain inflation pressure of the inflatable segments to control blood flow or to operate as an occlusive tourniquet.

9. The biosensory garment of claim 8 wherein the air supply includes an air pump.

10. The biosensory garment of claim 8 wherein the air supply includes a cylinder of compressed gas.

11. The biosensory garment of claim 1 wherein the central processor transmits information from the sensors to the command computer over a wireless personal network area protocol and wherein the command computer can comprise a remote server or smart device including a mobile phone, personal digital assistant, laptop or tablet computer.

12. The biosensory garment of claim 1 wherein there is one or more heating elements associated with the wearable portions to maintain or increase body temperature as required.

13. The biosensory garment of claim 1 wherein there is a portable rechargeable battery power supply to operate the one or more physiological sensors, the central processor, any transceiver, any heating element, any air supply or microprocessor controlled air valve.

14. The biosensory garment of claim 1 wherein the command computer coordinates and controls the operation of the one or more physiological sensors, any transceiver, or any heating element via the central processor.

15. The invention of claim 1 used for the ambulatory acquisition of vital signs and health status monitoring external to a clinical environment over extended time periods in the recovery and treatment of an injured wearer.

16. The invention of claim 1 used as a military garment to facilitate the acquisition of vital signs and health status of combatants during normal operational duties and wherein the vital signs and health status information can also be used to anticipate any necessary medical intervention or treatment by first responders or medical prior to rescue and evacuation of an injured combatant.

17. The invention of claim 1 used as an ambulatory sports activity and fitness monitoring device in the training and optimization of athletic performance and in the treatment and recovery of an athlete from a sports injury.
